(19) United States
(12) Patent Application Publication
(10) Pub. No.: US 2005/0283043 A1
(43) Pub. Date: Dec. 22, 2005

(54) Self-contained, submersible, autonomous, speaking android

(76) Inventor: Bradley G. Sisk, Washington, MI (US)

Correspondence Address: BRADLEY G. SISK, APT # 5, 545 SOUTH DIVISION, ANN ARBOR, MI, 48104, US

(21) Appl. No.: 10/701,985

(22) Filed: Nov. 6, 2003

(51) Int. Cl. A61F 5/00

(52) U.S. Cl. 600/38

(57) ABSTRACT

A self-contained anthropomorphic robot for erotic entertainment, stunt work, and commercial display. The robot has a contiguous, anatomically correct, lifecast outer skin. The outer skin is elastic, pigmented, and translucent, and is colored with superficial layers of translucent coloring agent to achieve a highly human appearance. In the preferred embodiment, the elastic skin is translucent silicone. This skin overlies soft padding and an articulated endoskeleton. The robot runs on its own internal power supply, and does not require an external power source. The robot can understand spoken commands and phrases, and can choose and speak its replies, according to internal logic. It generates its own body warmth, and is capable of operation under water.

### BACKGROUND-FIELD OF INVENTION

This invention relates to the field of robotics in general, and specifically to the field of anthropomorphic robots, also known as “androids”.

### BACKGROUND-DESCRIPTION OF PRIOR ART

The concept of a machine built in the image of a human being is as old as civilization, and populates the mythology of many cultures. The mythical golem, of slavic folklore, was an artificial man—built and animated by wizards, through sorcery. There are also references to animated mechanical creations in Greek mythology. Magically or mechanically-animated automatons (today called “robots”)—even those created in the image of human beings—are present in the mythology of many cultures. If man is seen as god's greatest creation, then the ultimate feat for technology might logically be creating a machine designed in man's own image.

The ancient Greeks crafted miniature “automatons” driven by falling water or springs. Though tiny, these were sometimes 2 dimensional figurines, sometimes abstract 3D representations of people or animals. They capable of one or two rudimentary motions, for amusement. Through the Renaissance period in Europe, numerous miniature human or animal figures were incorporated into spring-driven clocks, animated by the same clockwork mechanism which kept time. They often performed rudimentary motions at the start of each hour, analogous to the Cuckoo clock. These were limited to simple, jerky motions. They were bound to the clock which drove them..

By the machine age, industrialization brought some slightly more sophisticated animated puppets. These could be construed as the earliest robots. Nickelodeon arcade displays of larger humanoid puppets, with segments carved from wood, or cast in wax, driven by larger springs and gears. Later, electricity could be employed. Often these animated displays served as entertainment, appearing to deal tarot cards, or read a user's fortune. They were heavy, and immobile—anchored to the bulky machinery driving them.

The toy industry also helped in the advancement of anthrobotics—specifically, doll-makers. Edison invented an infant-sized doll, with a tiny, built-in phonograph, to effect speech. The face was rigid and fixed. It had a hard shell head and an abstractly human ragdoll body—like most baby dolls then and since. It was a mechanically-driven speaking doll. Sadly, the little records wore out after only 4 or 5 plays, so the toy flopped. Many dollmakers have since sought ways to animate their dolls. Perhaps the best and most relevant example of this In the prior art is U.S. Pat. No. 3,298,130 (Ryan, 1963). It also used a phonograph to playback speech. The ingenious design also actuated the eyes and lips to move (crudely) according to the phonograph pickup. But no blinking, no jaw movement. It was not electronic—a string had to be pulled each time by the user, this drove all the internal gears and clockworks to play the phonograph and move the eyes and lips. But this was the extent of the animation and the realism. Examples of similar animated baby dolls include U.S. Pat. No. 03,699,707(Sapkus, 1972), and U.S. Pat. No. 03,881,275 (Cogan, 1975). Each had a one or two of the defining features required by an android robot.

With the rapid advancement of electronics through the 20^{th} century, and their miniaturization, the field of robotics accelerated its advancement. Amusement parks and movie studios pioneered “animatronics”; lifelike animal, monster, or humanoid puppets, usually powered by high power hydraulics or AC. Walt Disney's theme parks erected early animatronic displays. The first attempt at high realism was made, and a high degree of artistic effort was invested. Animatronic figures which were quite convincing, from a distance. Though still relatively immobile, confined in their movement to a small area, they did appear to speak, emote, sing, dance, and interact with each other. With this approach to animatronics, one or more puppets was hooked-up to an external, immobile hydraulic, pneumatic, or electrical drive system. Individual puppets did not have an internal, self-contained drive system, and were not free to be carried or moved while in operation. In most cases, they were not true robots—in that they were not self-directed in their behavior. A human operator had to either set up a control program ahead of time, to execute a series of behaviors, or else cue the puppet in real-time, by remote control. It is important to make a technical distinction between puppets and robots. A puppet's actions are decided (ahead of time, or on-the-fly) by a human operator. In contrast, a robot makes its own behavioral decisions using a built-in logic. Some relevant animatronics patents include U.S. Pat. No. 0,327,7594 (Rogers et al., 1966), U.S. Pat. No. 3,912,694 (Chiappe, 1975). and U.S. Pat. No. 4,107,462 (Asija, 1978).

Hollywood film studios also contributed to the advancement—especially from the aesthetic end. But also in developing cheaper, lighter, and lower-power approaches. Special effects departments developed aesthetically-convincing puppets, using molding and casting techniques, glass eyes, and wigs. They mounted elastic skins over mechanical armatures. Low voltage DC actuators signaled by RF transmitters (for RC airplanes) replaced bulky hydraulic and AC cables. Again, these were not autonomous robots, but human-operated puppets.

With regard to true autonomous robots, some prior art patents outline designs which encompass one or a few of the defining characteristics of a true robotic android. U.S. Pat. No. 6,480,761, issued to Ueno, et al (2002). outlines a two-legged robot which walks bipedally, as humans do. However, this robot consists of a naked framework with only a rough bipedal shape. It was a clunky metallic armature, with no contiguous, anthropomorphic containing shell or skin. This robot had no fingers, no hands. It had no mouth, no pivotable jaw, and no facial features. The head was not shaped anatomically, it was just a sphere. Not intended as an anatomical model. It was not claimed to be capable of operation under water.

More relevant is U.S. Pat. No. 4,177,589 , issued to Villa (1979). a “Three dimensional animated facial control”—straddling the line between puppet and robot. This was an electronic device to animate a cast rubber face: opening and closing the mouth, moving the jaw, rounding or stretching the mouth, and drawing the lower lip inward relative to the upper lip in time with an audio signal. Pre-recorded audio could be fed into the device. It used analog circuits to detect specific waveforms corresponding to vowel sounds in the audio signal(analog parsing), and actuated the appropriate mouth and lip movements that a human being would make in speaking those sounds. With playback of the audio, the face appears to speak. Though limited in scope to just the mouth, this was an early attempt at mimicking facial expressions. It did not encompass blinking or eye movement. It was limited in scope to only a face. The design included no body. It was an analog signal-processor, not a robot executing behavioral software. Ultimately s human made the decision ahead of time as to which words would be spoken. There was no built-in decision logic.

Japanese engineers have patented many advances in vaguely humanoid, bipedal robots. Sony corp.'s much publicised “Assimo” prototype exemplifies most of these. U.S. Pat. No. 6,317,652 (Osada, Taku 1999) Outlines a legged, mobile robot, detailing approaches to maintaining balance and gait. Although a true robot, it's only vaguely humanoid in shape, with no molded, contiguous skin, or internal fluid bladders, or internal heating system. It does not cover speech, nor facial movements. No movement other than limb movement within its scope. It simply does not achieve convincing human appearance with speech. No claim is made as to its operation under water. Robots with this same morphology are outlined in U.S. Pat. Nos. 6,564,888, 6,493,606. From an American inventor, U.S. Pat. No. 6,532,400 details a biologically-inspired bipedal walker.(Jacobs, 2003). The design encompasses a pair of humanoid legs, and an abdominal section coordinating their movement. The design encompasses only a bare armature, anatomical realism is not the intent of this device. There is no corresponding upper body. No claim is made as to its functionality under water.

Conversely, U.S. Pat. No. 5,394,766 (Johnson, et al. 1995) comprises a legless robotic torso with nine degrees of freedom of rotation. Roughly humanoid in shape—with two arms and a thorax. Not an anatomical android, however, and no skin, facial features, warmth, or hair. Not specified as submersible.

Below is a list of other prior patents, less directly related to the field of the present invention.

| | |
|---|---|
| Pat 5,762,390 | Pat 5,753,834 |
| Pat 5,647,723 | Pat 6,512,965 |
| Pat 5,832,189 | |

### FEDERALLY SPONSORED RESEARCH

Not Applicable

### OBJECTS & ADVANTAGES

As a self-contained, submersible, autonomous, speaking android, the present invention has the following objects and advantages:
a) To provide an anthropomorphic robot which houses its own, internal power supply and drive system, and does not need to be connected to external equipment during operation.
b) To provide such a robot, with said internal power supply, also concealed in its outer skin, ports through which a user can periodically recharge said internal power supplies.
c) To provide an anthropomorphic robot which uses a hybrid low-voltage electric/pneumatic internal drive system.
d) To provide a robot which is genuinely self-directed and autonomous: deciding and executing its own actions according to software code and hardware carried on board—not requiring a human operator or remote control.
e) To provide a robotic device with a single, contiguous external skin shaped to match, with anatomical accuracy, a human being, including organic or synthetic hair follicles anchored into said skin.
f) To provide a robot with said flexible and translucent skin, painted with layers of translucent color, to appear convincingly human.
g) To include in said anthropomorphic robot, an internal heating system to simulate human body warmth.
h) To include in said robot, under the external skin, soft filling materials to replicate the compressibility and fluidity of human adipose and flesh.
i) To include in said robot, a jointed armature, abstractly modeling the human skeleton, and allowing for realistic (human-like)limb, neck, hand, and facial motions.
j) To provide such an autonomous robot, which can play back pre-recorded or synthesized audio, in synchrony with movement of its jaw, to simulate the effect of human speech.
k) To provide such a robot, encompassing all of the above objects, and also capable of operation submersed in water.
l) To provide such an anthropomorphic robot, including even molded body cavities and genitals of either gender, allowing for human sexual gratification.
m) To provide such an antropomorphic robot, for further use in film and video, taking the place of human stunt people in underwater scenes, and sparing them the risk.
n) To provide such an anthropomorphic robot, for use as dynamic target-training for police and armed forces.

As illustrated in the above review of relevant prior art, the above objects comprise advantages over prior art designs in the field of anthropomorphic robots. Among other advantages, the present invention overcomes prior art limitations, because it: carries its own self-contained power supply and drive system; while operating completely autonomously; while also having translucent layering of color for its skin, for highly convincing aesthetic realism; while also being self-contained in a single, contiguous, and flexible skin; while also being shaped to a fully anatomically-correct human likeness, including genitals; while also replicating human body warmth, while being capable of realistic limb, hand, neck, and facial movements; while also capable of jaw actuation in synchrony with playback of audio—to simulate speech; while also being capable of operation submersed in water.

### DETAILED DESCRIPTION

Obviously, the invention is shaped to resemble, as closely as possible, the human form. As an android robot, it is intended to be anthropomorphic. This is accomplished by its morphological system. The present invention Encompasses 5 anatomical systems. These are: the Morphological system; Endoskeletal system, the Electromotility system, the Pneumatic System, and the Thermal System. Each system serves a specific end in the design.

The Morphological System is responsible for giving the device a highly-convincing human likeness. As claimed, this encompasses shape,tactile feel, and color. The system consists of the outer mold-cast elastic skin, which is visible to the user—as well as the subdermal “fleshy” layers of foam rubber, hot melt vinyl, silicone rubber, silicone gel, and even water-filled bladders—which lie beneath the skin. The shape-memory properties of the cast rubber skin retain that shape for the product lifetime. Body hairs are embedded in the skin, and a detachable wig is afixed to the scalp. Plastic eyes, and a special layered airbrushing technique, layers of translucent paint, round out the surface aesthetics. This is how an authentic human appearance is achieved in the invention. The remainder of the morphological system exists to achieve tactile realism; to create the feeling of soft human flesh. A layer of soft foam rubber underlies the rubber skin, approximating the same compressibility and flexibility of human subcutaneous and adipose tissues. In more adipose areas, softer polymers and gels are also employed. And fluid-filled bladders fill out the feamle breasts and body cavities.

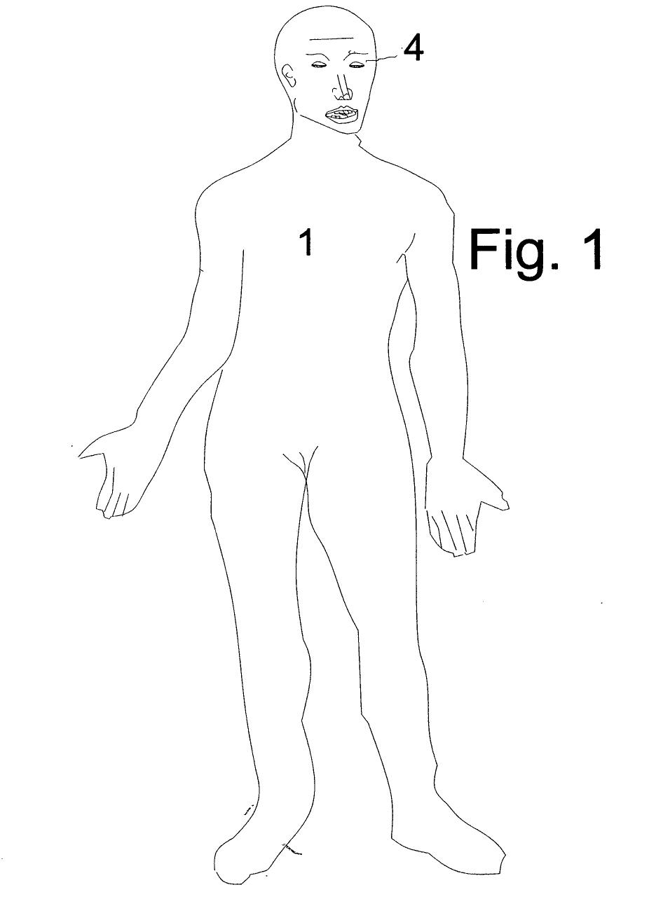

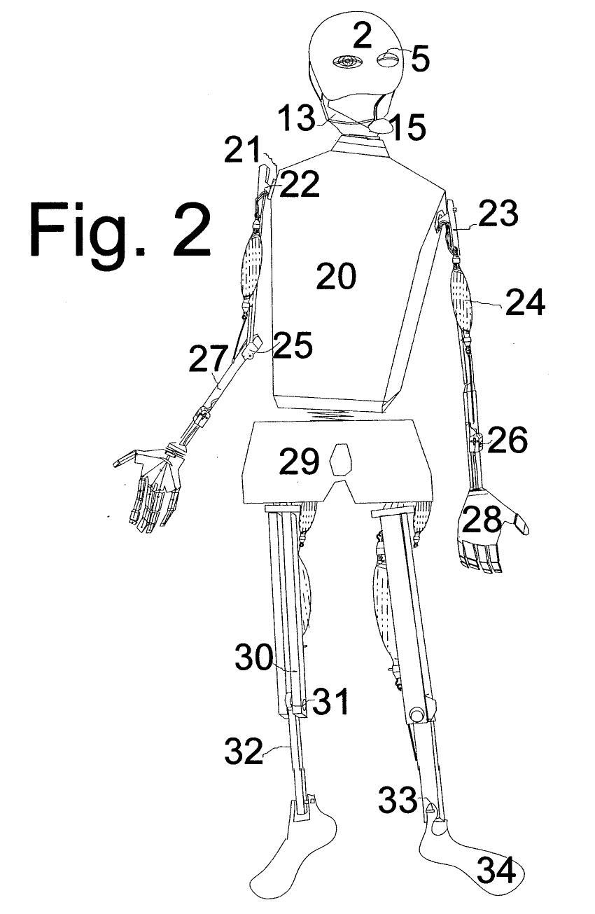

FIG. 1 is an illustration of the visible outer surface of the morphological sytem—the single, mold-cast skin. For anatomical correctness, gender-specific genitals are molded-in. So are the body cavities. Individual body hairs are permanently anchored into the skin, and a replaceable wig is attached to the scalp using a temporary fastener, such as hook-and-loop fabric strips. FIG. 2 is a cutaway view of the morphological system, showing the outer skin and underlying polymer layers and fluid bladders, in relation to the other component sytems.

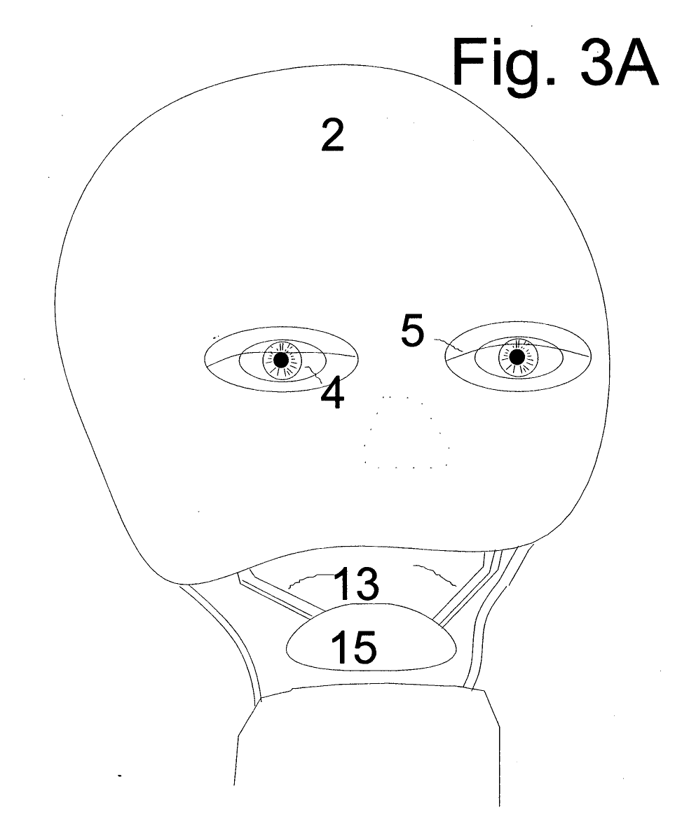

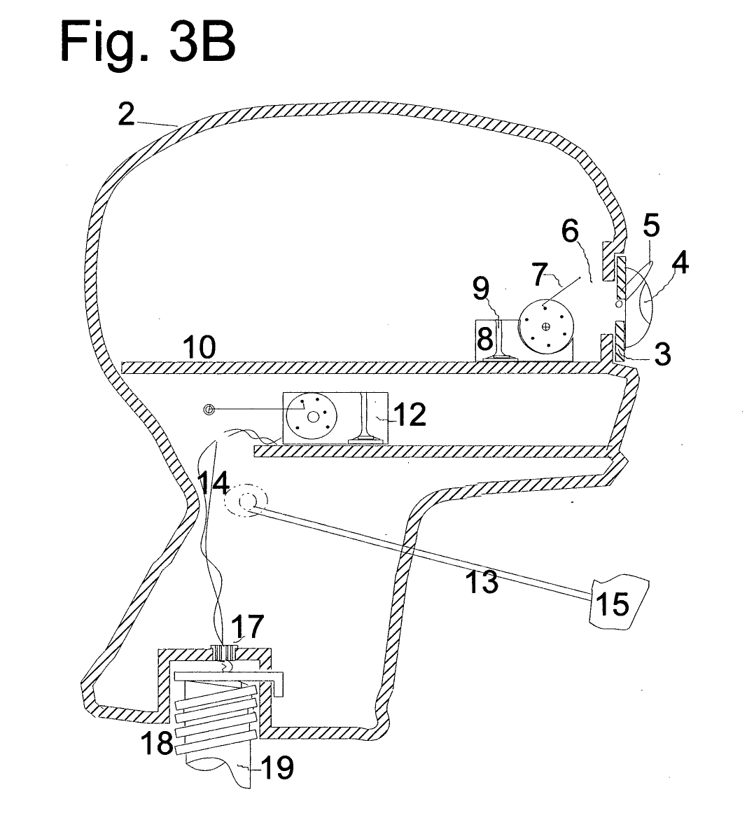

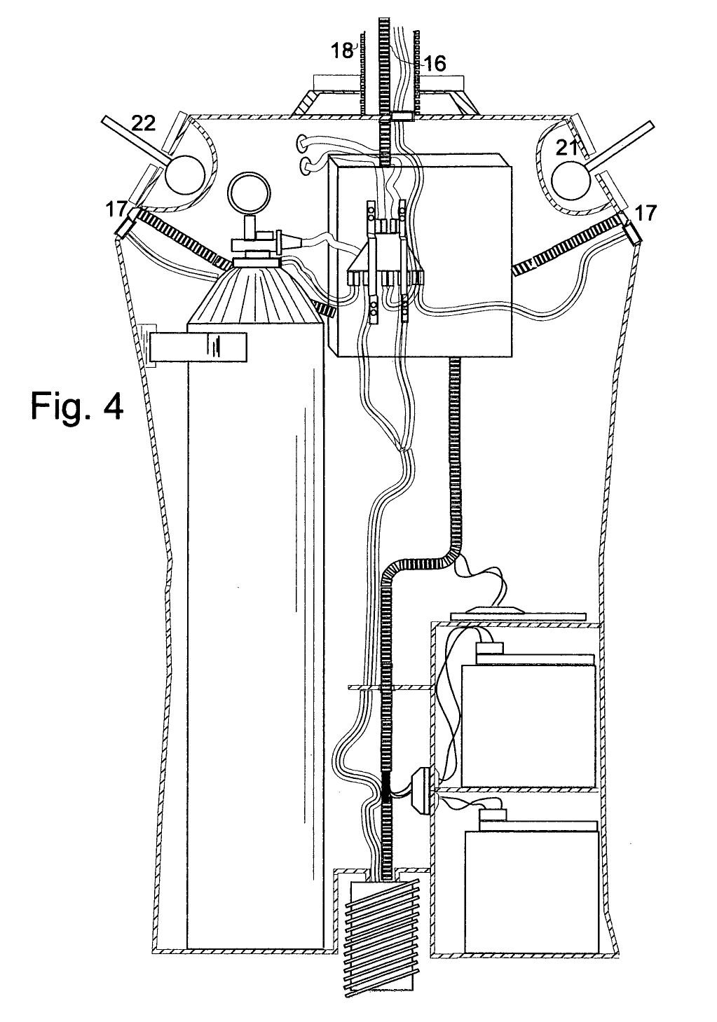

The endoskeletal system is diagramed in FIG. 3. An extreme simplification of the human skeleton, it is designed only to provide for realistic face and limb movement in the invention. The hands are further articulated to allow for opening and closing of the hand (FIG. 4).

The present embodiment makes no allowance for individual finger motion, all fingers contract or relax as a group, allowing only a simple clasping motion. The feet are constructed as solid units, they are not articulated as the hands are, but are just a continuation of the lower legs. There is no joint between the torso, the neck, or the skull. Instead, a flexible connecting element is used.

To allow for realistic limb movement, 3 types of joints are used. A ball-in-socket type joint is used at the shoulders, and hips. A plastic or teflon sphere sits inside a socket. The elbow joint is a simple hinge which permits the forearm to rotate in only one plane. The knee joints also allow only one rotational plane, but via a different design.

The torso and abdomen elements are constructed of a hollow shell, of a durable, rigid material. A cutaway Or cross-sectional view of is provided of the entire abdominal/thoracic region, diagramming the Arrangement of The internal parts. A single view has been magnified to facilitate viewing of the many parts, therefore this single cross-sectional view is spread out vertically across three separate pages. The appropriate illustrative technique has been used for this approach—in accordance.

The head is supported by the underskull, also diagrammed. The upper skull plate supports the scalp and face. To allow for realistic jaw movements, a separate segment—the mandible—is attached to it at the TMJ pivot joints on the left and right side of the face. It is comprised of a posterior mandible wire, which extends away from the joints, and holds the menton plate. The menton plate supports the chin, and prevents collapse of the chin inward. It is the segment which pushes the skin of the mouth to open when the jaw moves. The upper skull plate is also penetrated by the two eye sockets, in which 2 realistic plastic eyes are mounted. Wire eyelid hoops traverse the eyeball, anchored within a thin membrane continuation of the rubber surface skin (eyelids). The eyelid hoops are attached as levers on pivots to 2 internal servos. As the servos run, the eyelid hoops sweep up and down, pulling the eyelids (a continuation of the facial skin) up and down to simulate blinking. This mechanism is best explained with the ElectroMotive System, below.

The Electro-Motive system consists of the batteries, servos, actuators, electronic circuits, and wiring which allow for the android's various movements and speech. The present embodiment is not designed to stand-itself up, walk, or move itself across a distance. The present embodiment can only move its articulated segments: open/close hands, flex wrists, move arms and legs, open and close its mouth to simulate speech, and open and close its eyelids.

Androids and animatronic machines in prior art used AC, or high-power pneumatics and hydraulics. This limited their portability, as they had to be “plugged-in” to a fixed socket, hydraulic, or compressed-air drive to operate. The current invention is an improvement on prior art as it is entirely self-contained and portable. It can be switched-on and operated anywhere, without the need to be plugged-into a bulky drive system or socket. Apart from the need to occaisionally recharge batteries and tanks, it carries everything necessary to operate, inside itself. And no AC current is used.

As diagrammed, the central thoracic cavity houses the rechargeable batteries inside the battery compartment. The main DC power plugs into a solid-state connection built into the battery compartment bulkhead, rather than penetrating it. Electrical buses and air hoses traverse bariers by connecting to sold sockets on either side—not by simply feeding through holes. This ensures water cannot affect the electromotive or pneumatic systems when the device is operating under water. Electrical wires are bundled into protective plastic wire sheaths inside the robot. DC current is supplied by the rechargeable batteries, wired in series. It flows up though the thoracic cavity to The watertight brain module. This watertight, static-shielding container provide a a watertight housing for all of the electronic control hardware; it contains the electronic circuits (the “brains”) of the robot. The afferent Power bus feeds current to DC to DC converters to split it into power supplies of several different voltages. The brain module houses the behavioral software, Stored as digital code on EPROM chips. Pre-recorded audio clips are also stored here (words and sounds). The robot's behavior is directed by the PIC microcontroller chip. This chip ticks away compute cycles, reading all incoming data from sensors, and executing the behavioral software. The software allows the robot to decide what it will do at each tick of the clock. The microcontroller is hardware interfaced with its EPROM memory, audio circuits, the digital pneumatic manifold switcher, a pick-up microphone, and the circuits to signal all of DC servos distributed throughout the electromotive system. By deciding which air valves on the compressed-air manifold should open or close, the microcontroller can thus contract or relax any given pneumatic muscle, and so move an arm here or a leg there. Output leads from the microcontroller connect to Wires in the bus efferent from the brain module. Each of these efferent data wires ultimately connect to the signal terminal of a given DC servo. In this way, the microcontroller chip can signal the left hand to close, or an eyelid to blink, or to oscillate the mandible in synchrony with playback of a sequence of audio clips—thus creating the illusion of speech. As noted earlier, the brain module is watertight. But for added protection, much of the electronics in the brainbox are also encased inside a solid block of inert silicone rubber.

As explained, delicate motions of the face and hands are effected by electronic servos. For the more powerful motions of the larger segments like the limbs, pneumatic actuators are used for more convincing muscle action. This is the purpose of the Pneumatic System. An electronically-switchable valve manifold is directed by the behavioral electronics (via wire) to feed air from the tank to the appropriate hoses for the limb in question. With repeated limb movement, the reserve of compressed air is used up. An accessible inlet valve on the android allows one to recharge the tank from the outside, by connecting it to an air compressor. A standard “quick-connect” jack is employed. All of the air hoses are bi-channeled, so that

The spent air is collected from the air muscle, and routed back through the efferent channel, ultimately through the manifold to an exhaust port (located on the back) which traverses the skin. This allows spent air to escape the hermetically-sealed robot. To conserve electricity and air, all actuations are opposed by spring mechanisms. Thus, power is only drawn for joint movement in one direction. Thus, only one servo is needed for each joint, not two. Energy, weight, and cost are conserved.

The Thermal System constitutes an improvement upon prior art designs: allowing for greater authenticity, greater tactile realism over cold rubber animatronics. Akin to the human circulatory system, it consists of a branching network of fluid-filled tubes. The fluid is water, which the user can simply pour into a reservoir accessible underneath a discrete cap on the head surface. From the reservoir, several DC-powered Thermal nodes, in strategic body locations, use electricity to heat the water, and drive a small peristalsic pump. In concert with the flexing of the tubing caused by the android's own body motion, these nodes keep a gentle circulation of warm water circulating just under the flexible skin. This is how body warmth is simulated in the robot.

1. An autonomous anthropomorphic robot with a translucently-pigmented realistic skin and the ability to speak for use as a sex toy and human sexual partner.
